(19) United States
(12) Patent Application Publication
(10) Pub. No.: US 2013/0344477 A1
(43) Pub. Date: Dec. 26, 2013

(54) METHODS OF CAPTURING BINDABLE TARGETS FROM LIQUIDS

(71) Applicant: Adrian Parton, Cambridge (GB)

(72) Inventor: Adrian Parton, Cambridge (GB)

(73) Assignee: MATRIX MICROSCIENCE LIMITED, Carldsbad, CA (US)

(21) Appl. No.: 13/979,383

(22) PCT Filed: Jan. 6, 2012
(86) PCT No.: PCT/EP2012/050180

§ 371 Date: Sep. 6, 2013

(30) Foreign Application Priority Data
Jan. 13, 2011 (GB) 1100515.4

(51) Int. Cl. C12Q 1/24

(52) U.S. Cl. CPC C12Q 1/24
USPC 435/5; 435/7.32; 435/6.1; 435/309.1

(57) ABSTRACT

A bindable target such as a bacterial cell, virus, or molecule is captured from a liquid by contacting the liquid with magnetically attractable particles have an affinity for the target, and causing said particles to move repeatedly through said liquid to at least one solid support zone by attractive magnetic forces to capture the target onto said particles. The particles may be ferromagnetic, paramagnetic or superparamagnetic particles and may bear antibody, antibody binding fragments, a substance having an epitope capable of reacting in a specific manner with an antibody, an aptamer, a nucleic acid sequence or a nucleic acid analogue sequence, biotin, avidin or streptavidin. The particles may be moved back and forth in the liquid between separated solid support zones by attractive magnetic forces which attract the particles temporarily to different solid support zones in turn

The present invention relates to methods of capturing a bindable target from liquids containing said bindable target and to assay procedures involving said bindable target.

Whilst the present invention is of broad and general applicability, it has particular relevance to the problem of monitoring microorganisms in food and water and will be described with particular reference to these contexts.

Traditional methods for assaying the content of organisms in water and food such as cryptosporidium and Salmonella are time consuming and labour intensive.

A major problem in such assay procedures is that the organisms may be present in very low numbers in large volumes of liquid and must first be concentrated into a volume in which they are detectable.

Conventionally, water samples have been concentrated by passing large volumes of water through a cellulosic filter material which is then broken up and placed in a smaller volume of liquid in which it is agitated over a prolonged period with a view to releasing the captured organisms from the filter material. The proportion of the organisms present in the liquid samples which are captured by this way and successfully released from the filter material is relatively poor and the operation is prolonged taking typically about twenty-four hours to perform. The product of this procedure is a sample in which organisms are still very dilute.

Traditional methods to detect the presence of organisms in food require an enrichment/incubation period intended to allow for growth of these organisms. The increased number of the organisms present in the enriched sample aids detection and identification. However, the need for an enrichment period means the operation procedure is prolonged typically by about two days and makes quantitation difficult.

More recently, WO-A-95/31726 has provided a method for capturing a bindable target from a liquid, using magnetically attractable particles with an affinity for said bindable target. The method comprises attracting said particles to a solid support by magnetic forces and washing large volumes of liquid through or over the solid support bearing the magnetically attracted particles. The particles are then assayed for the captured bindable target. This method has been found to capture a substantial proportion of the bindable target present in the liquid. These can be released into a smaller volume of liquid, so concentrating them.

The present invention provides a method of capturing a bindable target from a liquid containing said bindable target, comprising contacting said liquid with magnetically attractable particles, which particles have an affinity for said bindable target, and causing said particles to move repeatedly through said liquid to at least one solid support zone by attractive magnetic forces to capture said bindable target onto said particles.

Bindable target is used herein to include microorganisms such as bacteria, protists and viruses, molecular targets such as antibodies and other proteins, peptides, nucleic acids, chemicals and ions, and cellular targets including fungal cells, plant cells, animal cells (including human cells), insect cells and stem cells.

Because the particles are moved repeatedly to at least one solid support zone, it is possible for the particles to track through large volumes of the liquid containing the bindable target to be captured during the time in which they are exposed to the liquid. The particles therefore may encounter and capture said bindable target in sufficient quantities for further operations to be carried out, even if the bindable target is present at low concentrations in the liquid.

Furthermore, as the particles can be held on a solid support zone while a liquid sample so treated is replaced with an as yet untreated liquid sample, multiple aliquots of the liquid may be treated by the same magnetic particles. This makes it possible for the volume of liquid containing the bindable target to be much greater than the volume of the liquid occupied by the particles during this operation.

It is preferable to release the magnetic particles with the captured bindable target from the final said solid support zone prior to the particles being assayed. This is preferably achieved by reducing the magnetic attraction, but may also be accomplished by vigorous washing or even air blasting whilst maintaining the magnetic attraction.

When the particles are released from the solid support, they may be collected in a much reduced volume of liquid. A very substantial concentration of the bindable target to be captured may therefore be achieved.

Alternatively, the particles may be assayed for the captured bindable target whilst retained on the final solid support zone.

The magnetically attractable particles may be ferromagnetic, paramagnetic or superparamagnetic particles.

Many forms of magnetically attractable particle are now known and easily commercially available. Examples include iron oxide particles as described in U.S. Pat. No. 4,554,088 and U.S. Pat. No. 3,917,538, nickel oxide particles as described in Biotec. and Bioengr. XIX: 101-124 (1977), Agarose-polyaldehyde beads containing magnetic particles as in U.S. Pat. No. 4,732,811, DYNAL beads (commercially available magnetic polystyrene coated beads); Magogel 44 (magnetic polyacrylamide-agarose beads), ENZACRY (poly-M-diaminobenzene/iron oxide) as described in Clin. Chim. Acta. 69:387-396 (1976). Cellulose containing ferric oxide particles are described in Clin. Chem. 26:1281-1284 (1980) and albumin magnetic microspheres as described in J. IMMUNOL. Methods 53:109-122 (1982). Magnetic porous glass particles are described in WO-A-93/10162.

Preferably the particles are of paramagnetic or superparamagnetic material as these particles do not clump together by virtue of acquiring a permanently induced magnetic field.

The particles may preferably have a specific binding affinity for the bindable target to be captured and for this purpose they may (depending on the nature of the bindable target to be captured) bear antibody molecules, antibody binding fragments, an aptamer, substances having an epitope capable of reacting in a specific manner with an antibody which may be present on the microorganism such as an antigenic protein or oligosaccharide, biotin, avidin or streptavidin, or like materials. They may bear a nucleic acid or nucleic acid analogue such as DNA, RNA or a synthetic analogue thereof. Also, the particles may have a chemical rather than a biochemical affinity for the bindable target to be captured. For instance, they may have chelating activity for capturing ions from the liquid.

They may have affinity for bacteria such as Salmonella, Listeria, E. col±0157 or Legionella or an affinity for parasites such as, cryptosporidium or giardia. However, the invention is of general applicability and may be used for capturing a wide range of bindable targets from a wide range of sample sources including body fluid samples such as blood, serum, salvia, urine, cerebrospinal fluid and so forth.

Preferably said liquid is contained within a container and wall portions of said container provide said at least one solid support zone.

Preferably said particles are moved repeatedly through said liquid between separated solid support zones by attractive magnetic forces which attract said particles temporarily to different solid support zones in turn.

The magnetic particles may be attracted to said solid support zone by use of a permanent magnet. Said magnet may be moved relative to said container to a position outside said container adjacent to said solid support zone to draw said particles to said solid support zone by attractive magnetic forces.

Said magnet may be moved relative to said container away from the exterior of said container to release said particles from said solid support zone by reduction of said magnetic forces.

A subsequent magnet may be applied to the exterior of said container at a different location from the previous magnet, to draw said particles through said liquid and onto a different solid support zone by attractive magnetic forces.

Alternatively, the magnetic particles may be attracted to said solid support zone by use of an electromagnet. Said electromagnet may be positioned outside said container adjacent to said solid support zone and may be activated to draw said particles to said solid support zone by attractive magnetic forces.

Said electromagnet may be deactivated to release said particles from said solid support zone by reduction of said magnetic forces.

A subsequent electromagnet, located at a different position outside said container from the previous magnet, may then be activated to draw said particles through said liquid onto a different solid support zone by attractive magnetic forces.

The most preferred container for said liquid is a tube with two vertical series of magnets arranged along the length of the tube on opposite sides of the tube.

The liquid containing the bindable target to be captured may be in motion. This motion preferably occurs in a transverse direction to the movement of the magnetic particles, so as to improve the capture of said bindable target. The flow of liquid may be reciprocating. When the liquid is in motion the particles travelling through the liquid are exposed to a vertical movement, thus creating a sine wave of movement and extending the flow path of the particles. This effect may be produced by gently pumping the liquid up and down the tube, e.g. by use of a syringe.

The invention includes labelling methods comprising capturing a bindable target to be assayed or used in an assay, by a method of capture as described above, and binding said bindable target captured on, or to be captured on said particles directly or indirectly to a detectable label. The label may be bound to the bindable target to be assayed before said bindable target is captured by the magnetically attractable particles or during or after the capturing of the bindable target.

Preferably, said label is bound to said bindable target via an immunological binding partner which binds selectively to said bindable target. Many different forms of detectable label are known in the art and in general any of these may be used, including fluorescent labels, luminescent labels, enzyme labels such as horse radish peroxidase, alkaline phosphatase, glucose oxidases, galactosidases or ureases, dye labels, phosphorescent labels, metal-chelating labels such as iminodiacetic acid, ethylenediaminetetraacetic acid, diethylenetriaminepentaacetic acid or desferrioxamine B, radio labels, spin labels, heavy metal labels, nucleic acid or nucleic acid analogue hybridisation labels, avidin or avidin like labels such as streptavidin, or biotin. Amongst these however the labels which are visually detectable, e.g. under the light microscope, are especially preferred.

The retention of the bindable target on a solid support zone via the magnetically attractable particles provides a ready way of separating excess label which may be washed away from the bound particles.

The invention also includes assay methods comprising capturing a bindable target to be assayed or to be used in an assay by a method of capture as described above, and conducting an assay of or using said captured bindable target. Optionally, the captured bindable target may be removed from the particles prior to or during said assay procedure.

The assay procedures involved may take a wide variety of forms including microbiology assay techniques such as staining or culturing, chemical assay procedures, enzyme assay procedures such as RIA or ELISA or nucleic acid procedures such as hybridisation assays.

The invention includes apparatus for use in capturing a bindable target from a liquid containing said bindable target comprising a container for containing a sample liquid which has on opposite sides a first magnet and a second magnet, and comprises means for repeatedly in alternation applying a magnetic field to the first side of the container by the use of said first magnet and then by the use of said second magnet applying a magnetic field to the second side of the container while simultaneously removing the magnetic field from the first side of the container.

Preferably, said first magnet and the second magnet are permanent magnets and said means comprises a support bearing said magnets on either side of said container, separated by more than the width of the container, the support being mounted relative to the container for relative movement to bring the first magnet and the second magnet alternately close to the container. Of course, either the magnets or the container may be moved to accomplish this.

Alternatively, said first and second magnet are both electromagnets which are activated and then deactivated in turn.

The invention will be further described and illustrated with reference to the accompanying drawings in which:

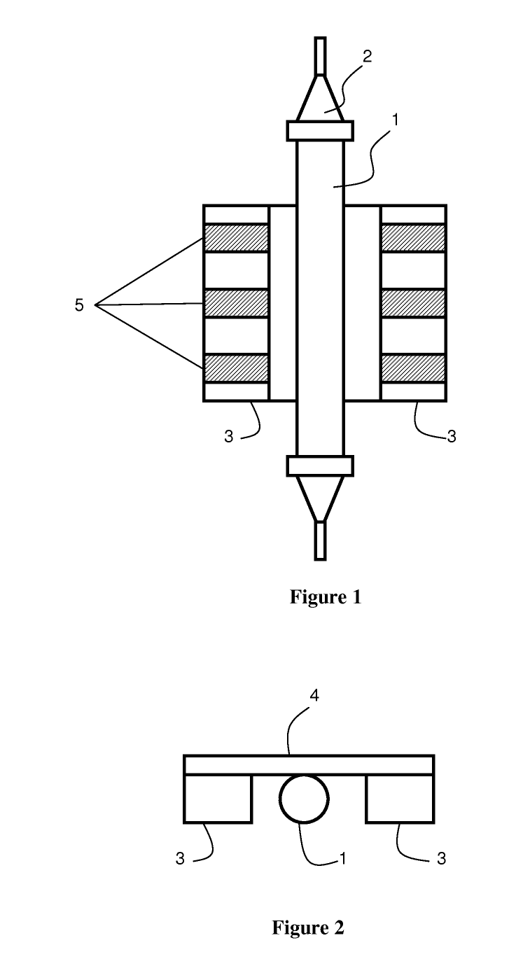

FIG. 1 shows a schematic vertical elevation of apparatus for use in the invention.

FIG. 2 shows a plan view of the apparatus shown in FIG. 1.

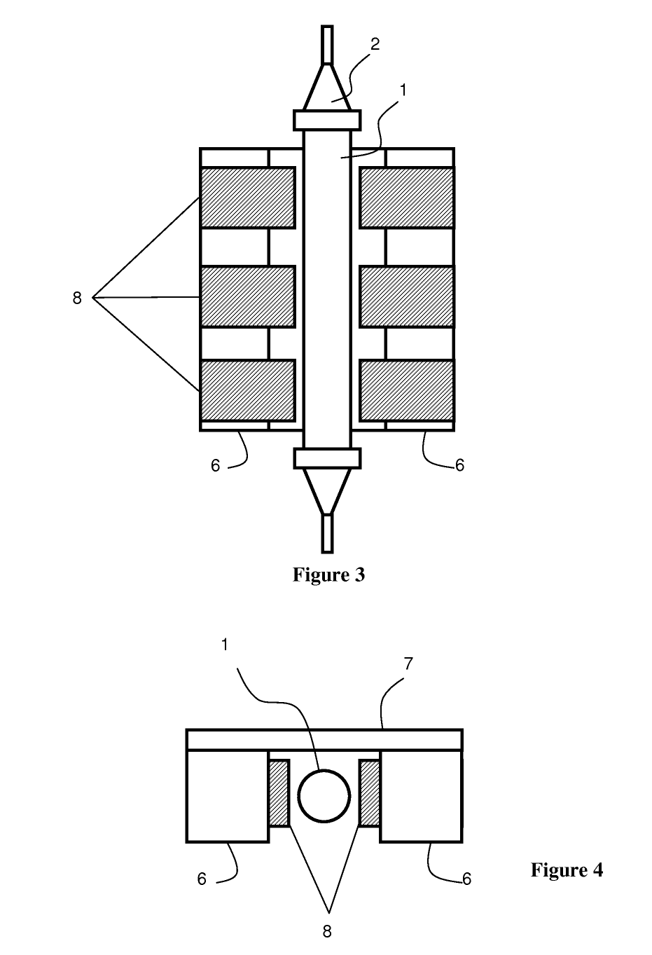

FIG. 3 shows a schematic vertical elevation of a second form of apparatus for use in the invention.

FIG. 4 shows a plan view of the apparatus shown in FIG. 3.

As shown in FIGS. 1 and 2, apparatus for use in the invention may comprise a container such as a tube 1, with a funnel connector 2 at each end, positioned in between two arms 3 of a magnetic cassette 4 each arm containing three permanent magnets 5 arranged in a vertical series along the arm of the cassette with their magnetic poles (either north or south independently) directed towards the tube.

The tube and the magnetic cassette are mounted relative to one another so that the tube can be adjacent to one arm of the magnetic cassette while simultaneously being remote from the other arm of the magnetic cassette.

The tube may be mounted fixed while the cassette is mounted to slide in a direction perpendicular to the length of the tube. The apparatus may also provide a means of driving the cassette motion, such as a mechanised device e.g. a solenoid or a reciprocating motor driven mechanical linkage.

Alternatively, the magnetic cassette may be mounted fixed, remaining stationary while the tube is mounted to move adjacent to each arm of the magnetic cassette in turn, possibly by means of the kind mentioned above.

In a typical procedure according to the invention, antibody coated magnetically attractable particles, optionally in a suitable buffer (e.g. PBS), are added directly to a liquid sample within the tube. The magnetic cassette is then moved to bring one magnet containing arm close to the wall of the tube. Over a period of seconds, the particles are drawn onto the solid support zone nearest to the applied magnetic field. Reverse motion of the magnetic cassette removes this applied magnetic field and the particles are permitted to detach from the solid support zone. As the second magnet containing arm of the magnetic cassette is brought close to the opposite side of the tube the released particles are drawn through the liquid sample to the solid support zone nearest to the subsequently applied magnetic field. This process is repeated, causing the magnetically attractable particles to move repeatedly between separated solid support zones.

An alternative form of apparatus shown in FIGS. 3 and 4 comprises tube 1 positioned in between two arms 6 of a magnetic cassette 7 each containing three electromagnets 8 arranged in a vertical series along the arm of the cassette with their magnetic poles (either north or south independently) pointing towards the tube. The electromagnets are activated and deactivated in series.

In use, activation of the first electromagnet draws the antibody coated magnetically attractable particles onto the solid support zone nearest to the applied magnetic field. Deactivation of this electromagnet removes the applied magnetic field and the particles are permitted to detach from the solid support zone. Activation of a second electromagnet draws the released particles through the liquid sample onto the solid support zone nearest to the second electromagnet. Each electromagnet is activated in turn.

Whilst side to side movement of the magnetic particles through the liquid is sufficient, one may add a vertical component to the movement in various ways. One option would be to make each individual magnet separately applicable, for instance by mounting each permanent magnet for separate sliding motion, whereby complex sequences of application of the magnets would become possible. For instance, one could apply first the magnet at the lowest position on one side, followed by the next higher magnet on the opposite side and so on to lift the particles through the liquid and then work back down to the starting position.

At the end of each procedure the external magnetic field is removed and the particles are permitted to detach from the solid support zones, optionally with agitation being used to disperse them. The particles may be run out of the tube for analysis, bearing any bindable targets which have been bound thereto. An advantage of this procedure is there is no need to use any chemical treatment to release the bindable targets from the solid support, which could affect the viability or integrity of the bindable targets. Chemical methods are, in contrast, normally needed in most immuno-affinity capture and release methods.

An alternative form of apparatus comprises a magnet and a container for containing a sample liquid such as a tube or beaker so that when in use the apparatus provides a means of repeatedly applying a magnetic field to a wall portion of the container and subsequently removing said magnetic field. The liquid sample may be shaken or inverted in between applications of the magnetic field, so as to improve mixing.

A further alternative form of apparatus for use in capturing a microorganism from a liquid containing said microorganism comprises a container for containing a sample liquid with two or more magnets arranged in a vertical series on opposite sides of the container, and means for moving a first vertical series of magnets adjacent to a first wall portion of the container and moving said second vertical series of magnets away from the container and means for subsequently moving said second vertical series of magnets adjacent to a second wall portion of the container while moving the first vertical series of magnets away from the first wall portion and continuing this process so as to bring each magnet adjacent to a wall portion in turn.

In any of the procedures above, the magnetic poles of said magnets may be pointing towards the container or may be aligned parallel to the container. When magnets with their magnetic poles aligned parallel to the container are brought adjacent to the container wall, a magnetic field is applied to two wall portions of the container, i.e. the wall portion adjacent to the north pole and the wall portion adjacent to the south pole.

The invention will also be further illustrated by the following examples.

### EXAMPLES

PATHATRIX® AUTO (WO-A-95/31726) is a well-established, validated Immuno-Magnetic Separation technique that has previously been shown to be more efficient at capturing bindable targets from liquids than other Immuno-Magnetic Separation techniques.

The Immuno-Magnetic Separation technique of the present invention was evaluated by comparing recovery of target microorganism directly against identical samples analysed using PATHATRIX® AUTO.

### Example 1

10 ml Buffered Peptone Water (BPW) samples were inoculated in duplicate at known levels with Salmonella Salford, Listeria monocytogenes or E. coli O157.

One sample from each duplicate was placed into a PATHATRIX® AUTO sample vessel and analysed using a standard 15 minute PATHATRIX® AUTO capture with a standard dose (50 μl) of antibody coated particles as appropriate to organism.

The remaining sample was placed into a 15 ml conical tube and an identical dose of paramagnetic particles (<1 μdiameter) coated with antibodies against either (a) Salmonella spp, (b) E. coli O157:H7, or (c) Listeria spp added directly into the sample.

The particles in this tube were pulled out of suspension and collected along the side of the tube using a series of magnets placed in a vertical line against the wall of the tube. The sample tube was then manually moved away from this set of magnets and another set directly placed onto the opposite side of the tube causing the particles to move through the sample.

The tube was moved between the sets of magnets once every 10 seconds and mixed by inverting once per minute. This procedure was carried out for a period of 15 minutes.

After the two capture procedures had been carried out, particles were captured from the samples using a magnetic rack and all liquid removed. Particles were then resuspended in 100 μl PBS.

Capture of target organism by the antibody-coated particles was assessed by spread plating the whole volume of particle suspension onto individual selective agar plates as appropriate to target organism.

Plates were inverted and incubated overnight at a temperature appropriate to target organism. After incubation, plates were removed from the incubator and examined for the presence of typical target colonies. All typical colonies present on selective agar plates were counted and recorded. (cfu=colony-forming units)

### Example 1(a)

### Salmonella Salford

| | |
|---|---|
| Inoculum level | Recovery (cfu) |

| (cfu/10 mls) | PATHATRIX AUTO | New Method |
|---|---|---|
| 520 | 33 | 59 |
| 5200 | 267 | 312 |
| 52000 | TNTC | TNTC |

### Example 1(b)

### E. coli O157

| | |
|---|---|
| Inoculum level | Recovery (cfu) |

| (cfu/10 mls) | PATHATRIX AUTO | New Method |
|---|---|---|
| 340 | 12 | 42 |
| 3400 | 478 | 528 |
| 34000 | TNTC | TNTC |

### Example 1(c)

### Listeria monocytogenes

| | |
|---|---|
| Inoculum level | Recovery (cfu) |

| (cfu/10 mls) | PATHATRIX AUTO | New Method |
|---|---|---|
| 2300 | 87 | 132 |
| 23000 | 579 | 721 |
| 230000 | TNTC | TNTC |

### Example 2

10 ml Buffered Peptone Water (BPW) samples were inoculated in duplicate at known levels with Salmonella Tranaroa or E. coli O157.

One sample from each duplicate was placed into a PATHATRIX® AUTO sample vessel and analysed using a standard 15 minute PATHATRIX® AUTO capture with a standard dose (50 μl) of antibody coated beads as appropriate to organism.

The remaining sample was placed into a 15 ml conical tube and an identical dose of paramagnetic particles (<1 μm diameter) coated with antibodies against either (a) Salmonella spp or (b) E. coli O157:H7 added directly into the sample. The beads in this tube were pulled out of suspension and collected along the side of the tube using a series of magnets placed in a vertical line against the wall of the tube. The sample tube was then manually moved away from this set of magnets and another set directly placed onto the opposite side of the tube causing the beads to move through the sample.

The tube was moved between the sets of magnets once every 5 seconds while a syringe was used to oscillate the liquid in the tube (one full oscillation every 2 seconds). This procedure was carried out for a period of 15 minutes.

After the two capture procedures had been carried out, beads were captured from the samples using a magnetic rack and all liquid removed. Beads were then resuspended in 100 μl PBS.

Capture of target organism by the antibody-coated beads was assessed by spread plating the whole volume of bead suspension onto individual selective agar plates as appropriate to target organism.

Plates were inverted and incubated overnight at a temperature appropriate to target organism. After incubation, plates were removed from the incubator and examined for the presence of typical target colonies. All typical colonies present on selective agar plates were counted and recorded. (cfu=colony-forming units)

### Example 2(a)

### Salmonella Tranaroa

| | |
|---|---|
| Inoculum level | Recovery (cfu) |

| (cfu/10 mls) | PATHATRIX AUTO | New Method |
|---|---|---|
| 98 | 6 | 17 |
| 980 | 43 | 136 |
| 9,800 | 157 | 306 |

### Example 2(b)

### E. coli O157

| | |
|---|---|
| Inoculum level | Recovery (cfu) |

| (cfu/10 mls) | PATHATRIX AUTO | New Method |
|---|---|---|
| 110 | 43 | 97 |
| 1,100 | 339 | 410 |

Because of sampling variations when measuring inoculum levels, the level of microorganisms actually present in the samples tested may not match the inoculum level stated, so the only valid comparisons that can be made are between the new method and PATHATRIX® AUTO applied to the same samples and not between Examples 1 and 2.

The Examples above show the method of the present invention to provide a better recovery rate of each microorganism tested as compared to PATHATRIX® AUTO, with the rate of recovery being particularly improved at low levels of inoculum.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

1. A method of capturing a bindable target from a liquid containing said bindable target, comprising contacting said liquid with magnetically attractable particles, which particles have an affinity for said bindable target, and causing said particles to move repeatedly through said liquid to at least one solid support zone by attractive magnetic forces to capture said bindable target onto said particles.

2. The method of claim 1, wherein said particles are ferromagnetic, paramagnetic or superparamagnetic particles.

3. The method of claim 1, wherein said particles bear antibody, antibody binding fragments, a substance having an epitope capable of reacting in a specific manner with an antibody, an aptamer, a nucleic acid sequence or a nucleic acid analogue sequence, biotin, avidin or streptavidin.

4. The method of claim 1, wherein said bindable target to be captured is a microorganism, a molecular target or a cellular target.

5. The method as of claim 1, wherein said bindable target to be captured is a bacterium, parasite or virus.

6. The method of claim 1, wherein said liquid is contained within a container and wall portions of said container provide said at least one solid support zone.

7. The method of claim 1, wherein said particles are moved repeatedly through said liquid between separated solid support zones by attractive magnetic forces which attract said particles temporarily to different solid support zones in turn.

8. The method of claim 1, wherein a magnet is moved to a position outside said container adjacent to said solid support zone to draw said particles to said solid support zone by attractive magnetic forces.

9. The method of claim 8, wherein said magnet is moved away from the exterior of said container to release said particles from said solid support zone by reduction of said magnetic forces.

10. The method of claim 8, further comprising application of a subsequent magnet to the exterior of said container at a different location from the previous magnet, drawing said particles through said liquid and onto a different solid support zone by attractive magnetic forces.

11. The method of claim 1, wherein an electromagnet in a position outside said container adjacent to said solid support zone is activated to draw said particles to said solid support zone by attractive magnetic forces.

12. The method of claim 11, wherein said electromagnet is deactivated to release said particles from said solid support zone by reduction of said magnetic forces.

13. The method of claim 11, further comprising activation of a subsequent electromagnet at a different position outside said container from the previous magnet, drawing said particles through said liquid and onto a different solid support zone by attractive magnetic forces.

14. The method of claim 1, wherein said liquid is contained within a tube and two vertical series of magnets are arranged longitudinally on opposite sides of the tube.

15. The method of claim 1, wherein said liquid is in motion in a transverse direction to that of the movement of the magnetic particles.

16. A labelling method comprising capturing a bindable target to be assayed or used in an assay, by a method as claimed in claim 1 and binding said bindable target captured on, or to be captured on, said particles directly or indirectly to a detectable label before and/or whilst said bindable target is captured on said particles on said at least one solid support zone.

17. An assay method comprising capturing a bindable target to be assayed or used in an assay, by a method as claimed in claim 1, and conducting an assay of or using said captured bindable target.

18. Apparatus for use in capturing a bindable target from a liquid containing said bindable target, said apparatus comprising a container for containing a sample liquid which container lies between on opposite sides a first magnet and a second magnet, and wherein said apparatus comprises means for repeatedly in alternation applying a magnetic field to the first side of the container by the use of said first magnet and then by the use of said second magnet applying a magnetic field to the second side of the container while simultaneously removing the magnetic field from the first side of the container.

19. Apparatus as claimed in claim 18 wherein said first magnet and the second magnet are permanent magnets and said means comprises a support bearing said magnets on either side of said container, separated by more than the width of the container, the support being mounted relative to the container for relative movement to bring the first magnet and the second magnet alternately close to the container.

20. Apparatus as claimed in claim 18 wherein said first and second magnets are electromagnets which are activated and then deactivated in turn.
